(19) United States
(12) Patent Application Publication
(10) Pub. No.: US 2022/0087660 A1
(43) Pub. Date: Mar. 24, 2022

(54) BIOPSY DEVICES, SYSTEMS, AND METHODS

(71) Applicant: Boston Scientific Scimed, Inc., Maple Grove, MN (US)

(72) Inventors: Andrew J. Schaubhut, Bolton, MA (US); James J. Scutti, Norwell, MA (US); Laura E. Christakis, Framingham, MA (US); Steven Delfosse, Waltham, MA (US); Laura E. Richards, Worcester, MA (US); Amanda Klinker, Vernon, CT (US)

(21) Appl. No.: 17/474,421

(22) Filed: Sep. 14, 2021

Related U.S. Application Data
(60) Provisional application No. 63/081,483, filed on Sep. 22, 2020.

(51) Int. Cl. A61B 10/02

(52) U.S. Cl. CPC A61B 10/0283; A61B 10/0275

(57) ABSTRACT

Various embodiments are generally directed to biopsy devices, systems, and methods for tissue sample acquisition, such as during fine-needle aspiration. Several embodiments are particularly directed to biopsy devices with features to facilitate accessing, acquiring, and/or retaining one or more tissue samples from a target biopsy site. In one embodiment, for example, a biopsy device may include an elongate member having an insert disposed within a lumen of an elongate member. The insert may be configured to mate with a needle disposed in the lumen to maintain a rotational orientation of the needle. In another embodiment, a needle may include multiple openings for acquiring a tissue sample. In yet another embodiment a helical component may be disposed in a needle lumen to acquire and retain tissue samples.

### RELATED APPLICATIONS

The present application is a non-provisional of, and claims the benefit of priority under 35 U.S.C. § 119to, U.S. Provisional Application Ser. No. 63/081,483, filed Sep. 22, 2020, the disclosure of which is incorporated herein by reference in its entirety.

This application relates to, and incorporates by reference in its entirety for all purposes, U.S. patent application Ser. No. 16/875,395, titled “Medical imaging devices, systems, and methods”, and filed on May 15, 2020.

### FIELD

The present disclosure relates generally to medical devices, systems, and methods. In particular, the present disclosure relates to biopsy devices, systems, and methods.

### BACKGROUND

Biopsies are a group of medical diagnostic tests used to determine the structure and composition of tissues or cells. In biopsy procedures, cells or tissues are sampled from an organ or other body part to permit their analysis, for example under microscope. If an abnormality is found through superficial examination such as palpation or radiographic imaging, a biopsy can be performed to determine the nature of the suspected abnormality. Generally, biopsies can be classified as either an excisional biopsy or an incisional biopsy. Excisional biopsies may include removal of an entire nodule or suspicious area. An incisional biopsy, on the other hand, may include sampling a portion of the abnormal tissue without attempting to remove the entire lesion or tumor. Incisional biopsies are typically safer and less traumatic than excisional biopsies. In one type of incisional biopsy, fine-needle aspiration (FNA), a sample of tissue or fluid is removed with a needle in such a way that cells are removed without preserving the histological architecture of the tissue cells. Typically, the needle in FNA is inserted through the working channel of an endoscope to access the target site and obtain a tissue sample.

It is with these considerations in mind that a variety of advantageous medical outcomes may be realized by the devices, systems and methods of the present disclosure.

### SUMMARY

In one aspect, the present disclosure relates to a biopsy device comprising an elongate member and an insert. The elongate member may have a proximal portion, a distal portion, and a lumen. The lumen may include proximal and distal openings and may extend from the proximal portion to the distal portion of the elongate member. The insert may be disposed in the lumen. The insert may include a lumen interface and a needle interface. The insert may be configured to mate with an insert interface of a needle disposed in the lumen and maintain a rotational orientation of the needle.

In some embodiments, the insert limits proximal or distal motion of the needle disposed in the lumen. In various embodiments, the insert interface comprises a flat surface generated by removal of material from a curved surface of the needle. In many embodiments, the device comprises the needle and the insert interface of the needle is configured to slide along the needle interface of the insert when the needle and insert are mated. Many such embodiments include a polymer stylet disposed within the needle. In some such embodiments, the needle is configured to slide longitudinally when the insert interface is mated with the needle interface. In various such embodiments, the needle interface of the insert extends a first length along a longitudinal axis of the elongate member and the insert interface of the needle extends a second length along the longitudinal axis of the elongate member, wherein the first length is less than the second length. In several embodiments, the lumen interface comprises a curved surface and the needle interface comprises a flat surface. In multiple embodiments, the needle interface comprises one or more of a channel, a groove, and a zig-zag. In some embodiments, the needle interface comprises a channel extending along a longitudinal axis of the elongate member. In various embodiments, the elongate member comprising a second lumen, wherein the distal opening of the lumen is orthogonal to a distal opening of the second lumen. In many embodiments, the device comprises the needle, the needle including a needle lumen with first and second openings, wherein the first and second openings are in a distal half of the needle. In many such embodiments the first opening of the needle lumen is orthogonal to the second opening of the needle lumen. Some such embodiments include a sheath disposed around a portion of the needle, wherein the sheath covers the first opening of the needle lumen. In various such embodiments, the needle comprises a scoop disposed adjacent to the first opening.

In another aspect, the present disclosure relates to a system comprising an elongate member, an insert, and a needle. The elongate member may have a proximal portion, a distal portion, and a lumen. The lumen may include proximal and distal openings and may extend from the proximal portion to the distal portion of the elongate member. The insert may be disposed in the lumen. The insert may include a lumen interface and a needle interface. The needle may be disposed in the lumen and include an insert interface. The needle interface and the insert interface may include corresponding mating features.

Some embodiments include a stylet disposed in a lumen of the needle. In some such embodiments, the stylet comprises a polymer. In various embodiments, the needle may include a needle lumen with first and second openings in a distal half of the needle. In various such embodiments, the first opening of the needle lumen is orthogonal to the second opening of the needle lumen.

In yet another aspect, the present disclosure relates to a method. The method may include one or more of: forming an insert with a needle interface; forming a needle with an insert interface, wherein the needle interface of the insert corresponds to the insert interface of the needle; mating the needle interface with the insert interface; and disposing the insert and the needle in a lumen of an elongate member.

In some embodiments, the method may include one or more of: forming a stylet from a polymer and disposing the stylet in a lumen of the needle. In various embodiments, the method includes press-fitting the insert into the lumen. In many embodiments, the method includes grinding the needle to form the insert interface. In several embodiments, the method includes pressing the needle to form the insert interface. In multiple embodiments, the method includes forming the insert by stamping. In some embodiments, the method includes disposing the insert and the needle in the lumen with press fitting. In various embodiments, the method includes disposing the insert and the needle in the lumen with thermal fitting.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIGS. 1A-1E illustrate various aspects of a distal portion of a biopsy device according to one or more embodiments disclosed hereby.

FIGS. 2A-2C illustrate various aspects of an exemplary elongate member for a biopsy device according to one or more embodiments disclosed hereby.

FIGS. 3A-3D illustrate exemplary inserts and needles for a biopsy device according to one or more embodiments disclosed hereby.

FIG. 4 illustrates various aspects of an exemplary elongate member for a biopsy device according to one or more embodiments disclosed hereby.

FIGS. 5A and 5B illustrate various aspects of an exemplary needle for a biopsy device according to one or more embodiments disclosed hereby.

FIG. 6 illustrates a distal portion of an exemplary needle for a biopsy device according to one or more embodiments disclosed hereby.

FIG. 7 illustrates a distal portion of an exemplary needle for a biopsy device according to one or more embodiments disclosed hereby.

FIGS. 8A-8D illustrate various aspects of an exemplary needle for a biopsy device according to one or more embodiments disclosed hereby.

### DETAILED DESCRIPTION

Various embodiments are generally directed to biopsy devices, systems, and methods for tissue sample acquisition, such as during fine-needle aspiration. Several embodiments are particularly directed to biopsy devices with features to facilitate accessing, acquiring, and/or retaining one or more tissue samples from a target biopsy site. In one embodiment, for example, a biopsy device may include an elongate member having an insert disposed within a lumen of an elongate member. The insert may be configured to mate with a needle disposed in the lumen to maintain a rotational orientation of the needle. In another embodiment, a needle may include multiple openings for acquiring a tissue sample. In yet another embodiment a helical component may be disposed in a needle lumen to acquire and retain tissue samples. These and other embodiments are described and claimed.

Some challenges in tissue sample acquisition include reliably obtaining a viable sample (e.g., sample may be too small and/or not from the biopsy target site), preventing needles from breaking or kinking, and controlling orientation and/or angle of attack of the needle. For example, a target site may be small and difficult to reach (e.g., located in the distal reaches of the lungs). Oftentimes a needle needs to be removed from a patient and reinserted after taking, or attempting to take, each sample. Further, adjusting the angle of attack of a needle can require repositioning of an endoscope the needle is disposed in. Either of which may lead to excessively invasive procedures with longer recovery times. Adding further complexity, needles can be fragile and prone to damage, such as kinking. Many such needles are required to largely remain in protective sheaths or catheters. These and other factors may result in biopsy devices and methods with limited capabilities, resulting in reduced applicability, poor adaptability, and limited functionality. Such limitations can drastically reduce the quality and usability of the biopsy devices, contributing to poor user experiences and adverse patient outcomes.

Accordingly, various embodiments of the present disclosure include biopsy devices with features to facilitate accurate and reliable accessing, acquiring, and/or retaining of one or more tissue samples from a target biopsy site. In many embodiments, one or more of the features may control the orientation needles disposed in lumens. In various embodiments, one or more of the features may enable adjusting the angle of attack of a needle and/or taking multiple samples without removal from a body lumen, resulting in more efficient and/or reliable biopsy procedures. For instance, being able to take tissue samples over a range or area without repositioning the elongate member and/or a bronchoscope the elongate member is disposed in can directly reduce patient trauma and improve outcomes. In several embodiments, one or more of the features may provide additional strength and/or durability to needles, such as via a stylet or a solid bore. In some embodiments, one or more of the features may improve the quantity and/or quality of tissue samples, such as via bidirectional tissue acquisition. In these and other ways, components/techniques described herein may improve biopsy devices.

It may be understood that the disclosure included herein is exemplary and explanatory only and is not restrictive. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Although endoscopes and endoscopic systems are referenced herein, reference to endoscopes, endoscopic systems, or endoscopy should not be construed as limiting the possible applications of the disclosed aspects. For example, the disclosed aspects may be used in conjunction with duodenoscopes, bronchoscopes, ureteroscopes, colonoscopes, catheters, diagnostic or therapeutic tools or devices, or other types of medical devices or systems.

Reference is now made to the drawings, wherein like reference numerals are used to refer to like elements throughout. In the following description, for purpose of explanation, numerous specific details are set forth in order to provide a thorough understanding thereof. It may be evident, however, that the novel embodiments can be practiced without these specific details. In other instances, well known structures and devices are shown in block diagram form to facilitate a description thereof. The intention is to cover all modification, equivalents, and alternatives within the scope of the claims.

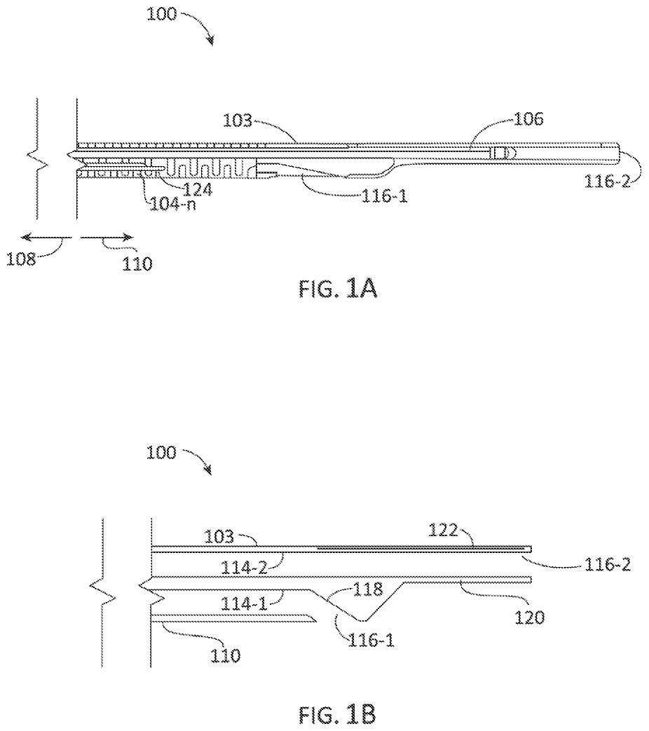

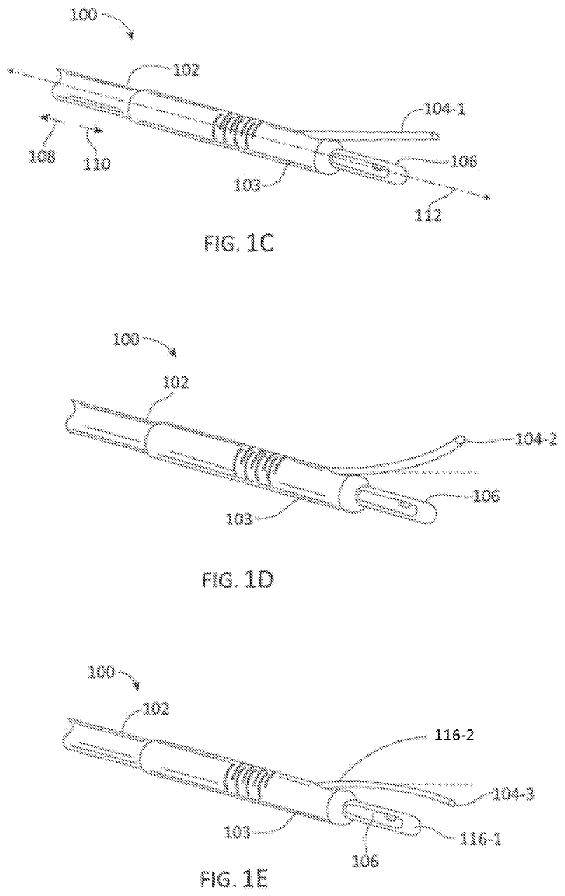

FIGS. 1A-1E illustrate various aspects of a distal portion 110 of a biopsy device 100 according to one or more embodiments of the present disclosure. More specifically, FIG. 1A includes a cross sectional view of the biopsy device 100 including probe 103 with a needle 104-n disposed therein. FIG. 1B includes a cross sectional diagram of the probe 103 of biopsy device 100. FIG. 1C illustrates a perspective view of biopsy device 100 including needle 104-1, elongate member 102, and probe 103. FIG. 1D illustrates a perspective view of biopsy device 100 including needle 104-2, elongate member 102, and probe 103. FIG. 1E illustrates a perspective view of biopsy device 100 including needle 104-3, elongate member 102, and probe 103. In various embodiments, needles 104-1, 104-2, 104-3 may provide three different angles of attack for acquisitions of tissue samples. The needles 104-1, 104-2, 104-3, 104-n (or needles 104) may be selectively disposed in a first lumen 114-1 of the elongate member 102. An imaging device such as an ultrasound catheter 106, may be disposed in a second lumen 114-2 of the elongate member 102. In some embodiments, a distal portion 110 of the biopsy device 100 may be inserted through a working channel of a bronchoscope to access a target site for tissue acquisition. FIGS. 1A-1E may include one or more components that are the same or similar to one or more other components of the present disclosure. Further, one or more components of FIG. 1A-1E, or aspects thereof, may be incorporated into other embodiments of the present disclosure without departing from the scope of this disclosure. Embodiments are not limited in this context.

One or more biopsy devices disclosed hereby may include one or more needles having predetermined shapes when in a deployed configuration that provide one or more angles of attack for acquiring tissue samples. More generally, the biopsy device 100 may include an elongate member 102 with a proximal portion 108 and a distal portion 110. In many embodiments, the elongate member 102 may include a dual lumen catheter. In many such embodiments, a needle for FNA may be disposed in the first lumen and an imaging transducer. The distal portion 110 of elongate member 102 may include a probe 103. The probe 103 may include distal openings 116-1, 116-2. The illustrated embodiment in FIG. 1A, includes a needle 106-n extending proximate the distal opening 116-1 and ultrasound catheter 106 extending proximate the distal opening 116-2.

FIGS. 1C-1E each include a needle 104-1, 104-2, 104-3, respectively, in the deployed configuration (i.e., extending out of the distal opening 116-2). Each of the needles 104 has a different deployed configuration, resulting in different angles of attack. For example, needle 104-1 may have a neutral angle of attack, needle 104-2 may have a positive angle of attack, and needle 104-3 may have a negative angle of attack. This may be accomplished via needles with arcs varying from negative to positive. The multiple angles of attack may allow different portions of a nodule to be biopsied. More generally, the multiple angles of attack may enable tissue samples to be acquired from an entire area or strip. Further, being able to take tissue samples over a range or area without repositioning the elongate member and/or a bronchoscope the elongate member is disposed in can directly reduce patient trauma and improve outcomes.

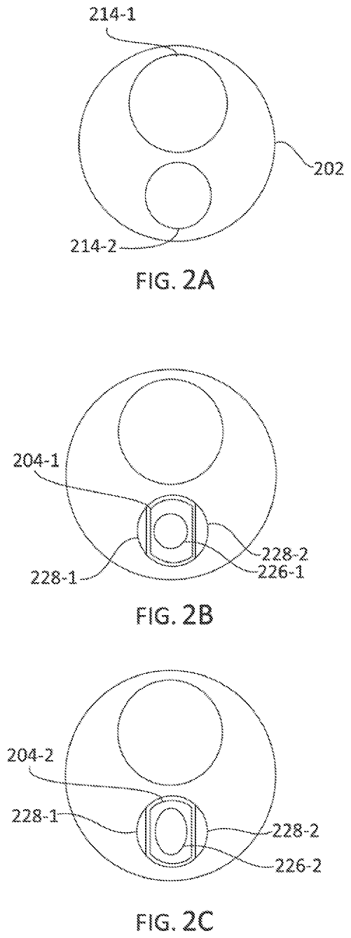

When the ends of the needles 104-1, 104-2, 104-3 are within the lumen 114-2, the needles can be in a common retracted configuration (i.e., aligned with longitudinal axis 112 of the elongate member 102), such as shown in needle 104-n in FIG. 1A. In various embodiments, shape memory materials may facilitate transitioning between retracted and deployed configurations. As will be discussed in more detail below, such as with respect to FIGS. 2A-4, the orientation of the needle may be controlled via inserts disposed in the lumen. Orientation control can facilitate needles bending in predetermined directions with respect to the opening 116-1.

In the illustrated embodiment, the probe 103 includes a ramp 118. The ramp 118 may adjust the angle of attack of needles 104. For example, the ramp 118 may facilitate a radial offset of the contact site for a needle and a target site, such as with eccentric nodules. In some embodiments, the needles 104 may include one or more compound bends or bend sections. For example, a first bend in a first direction and a second bend in a second direction. In several embodiments, one or more of the ramp 118 and the needles 104 facilitate customization of the angle of attack among other characteristics (e.g., contact site offset in one or more dimensions).

In some embodiments, probe 103 may comprise an endcap coupled to the distal end of elongate member 102. In other embodiments, probe 103 may comprise a portion of the elongate member 102. For example, the probe may comprise distal portion 110 of the elongate member 102. Further, the probe may be integrally formed with the elongate member or formed separate from the elongate member. More generally, the probe may refer to the distal end of a biopsy device that is inserted into a body lumen.

One or more components disclosed hereby may be constructed from an elastomer and/or a polymer (e.g. polycarbonate, acrylonitrile butadiene styrene (ABS), high-density polyethylene (HDPE), Nylon, polyether ether ketone (PEEK), silicone, thermoplastic, plastic, or the like). Various components disclosed hereby may be constructed from a metal (e.g., stainless steel, titanium, aluminum, alloys, or the like). Some components disclosed hereby may include one or more shape-memory materials (e.g., nickel titanium alloy (nitinol)). For example, bends or bend sections in needles may comprise nitinol. In many embodiments, the reduced flexural strength of various shape-memory materials may be utilized, such as to prevent deformation due to ramp 118.

Referring specifically to FIG. 1A, the needle 104-n may include a stylet 124. The stylet may provide additional part integrity to resist damage, such as deformation. In many embodiments, the stylet 124 may comprise a composite and/or polymer material, such as an engineering plastic. In various embodiments, the stylet 124 may function as an extraction member and ejecting tissue samples from the needle lumen. In some embodiments, a needle may comprise or refer to a hypotube. In one or more embodiments, a needle sheath with tight tolerances with respect to a needle may ensure sufficient column strength for the needle to puncture a target site, such as a pulmonary nodule.

Turning to FIG. 1B, the probe 103 includes an imaging window 120 and a marker 122. In many embodiments, imaging window 120 may refer to one or more portions of the probe 103 that are substantially transparent to the imaging energy wave lengths (e.g., from ultrasound catheter 106) while marker 122 may refer to one or more portions of the probe that are relatively opaque to the imaging energy wave lengths. Marker 244 may comprise any medium that absorbs imaging energy wavelengths (e.g., ultrasound waves). For example, metal or metal alloys (e.g., stainless steel or nitinol) may be used. In some embodiments, non-metals may be used, such as air pockets embedded in the wall of the imaging window.

In various embodiments, the marker 122 may be radiopaque, such as to show up on x-ray and/or fluoroscopic imaging additionally, or alternatively. In some embodiments, marker 122 may be positioned to indicate in a generated image where a needle would be positioned in the deployed configuration. In several embodiments, the probe 103 may be positioned based on the generated images, the elongate member 102 may be axially rotated to cause probe 103 to rotate, repositioning the distal opening 116-2. For example, a handle assembly coupled to a proximal end of the elongate member 102 may be rotated to align the distal opening 116-2 with a target nodule based on indications of marker 122 in generated images. In some such examples, once aligned, one or more needles may be used to contact and/or penetrate the target nodule. In various embodiments, a marker may be embedded in a wall of a lumen, such as the wall of the second lumen 114-2. As will be appreciated, device rotation (e.g., orientation of the marker and the needle radially) may enable more efficient biopsying of eccentric nodules, e.g., when biopsying target tissue that has irregular margins, is of an asymmetric shape, does not extend around an entire circumference of the body lumen, and the like, where control or orientation and position of the needle may be more critical.

As an example, marker 122 may be oriented around the circumference of the imaging window at a known angle from distal opening 116-1. In such a case, e.g., when targeting a lung nodule for core biopsy, marker 122 may be oriented on the radial ultrasound image at the known angle from the intended biopsy site (e.g., an offset angle), so that a needle exiting distal opening 116-1 will be correctly aligned with the biopsy site (orientation control of the needles may additionally assist in this). In a further such example, the marker 122 may be oriented on the radial ultrasound image 180 degrees across from the intended biopsy site. In many embodiments, the known angle from the intended biopsy site may be configured such that tolerances may be provided. For example, the marker 122 may be oriented on the radial ultrasound image 180 ±35 degrees from the intended biopsy site. Utilizing an offset angle can prevent the marker 122 from blocking direct imaging image of a target site while still providing indications of where a needle will contact/puncture a target site.

FIGS. 2A-2C illustrate various aspects of an elongate member 202 according to one or more embodiments of the present disclosure. More specifically, FIG. 2A illustrates a transverse cross-sectional view of elongate member 202 with lumens 214-1, 214-2. FIG. 2B illustrates inserts 228-1, 228-2 (or inserts 228) and needle 204-1 with needle lumen 226-1 disposed in lumen 214-2. FIG. 2C illustrates inserts 228 with needle 204-2 with needle lumen 226-2 disposed in lumen 214. In various embodiments, the inserts 228 in conjunction with features of the needles 204, an orientation of the needle may be maintained as it is moved proximally and distally in the elongate member 202. In several embodiments, the inserts 228 in conjunction with features of the needles 204 may limit a range of movement of the needle in one or more of the proximal and distal directions. For example, the distance the needle can be proximally extended out of the lumen 214-2 may be limited. In some embodiments, FIGS. 2A-2C may include one or more components that are the same or similar to one or more other components of the present disclosure. Further, one or more components of FIG. 2A-2C, or aspects thereof, may be incorporated into other embodiments of the present disclosure without departing from the scope of this disclosure. For example, needle 204-1 and/or needle 204-2 may be the same or similar to one or more of needles 104. Embodiments are not limited in this context.

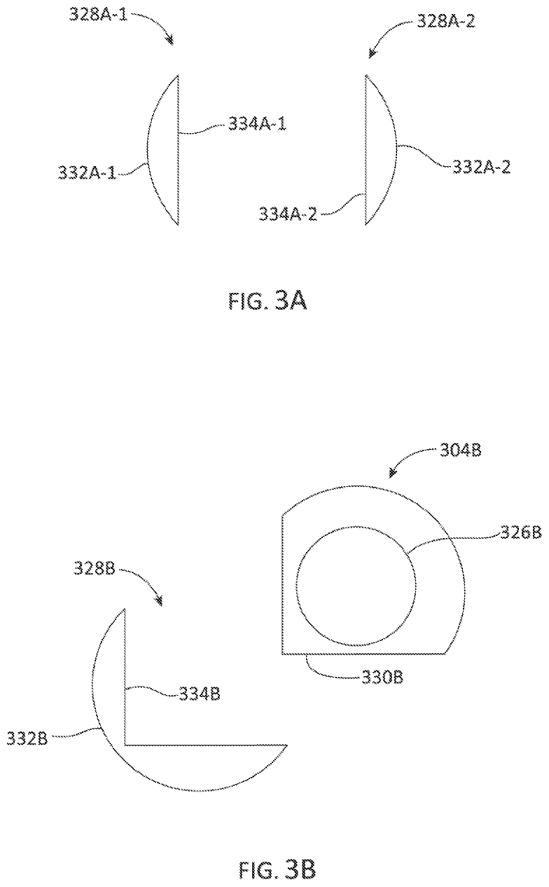

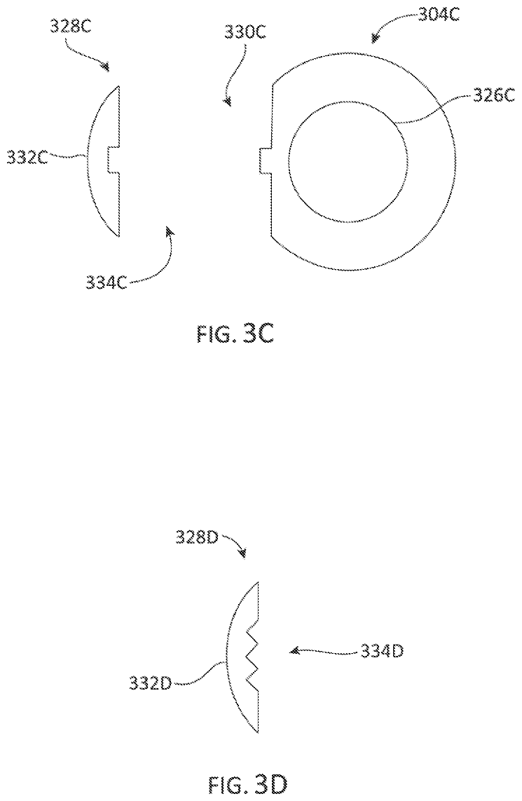

In various embodiments, the needles may include one or more surfaces that interface with one or more inserts to maintain orientation of the needles. Referring to FIG. 2B, the needle 204-1 includes a first surface that interfaces with insert 228-1 and a second surface that interfaces with insert 228-2. Similarly, referring to FIG. 2C, the needle 204-2 includes first and second surfaces that interfaces with insert 228-1 and a second surface that interfaces with insert 228-2. As will be described in more detail below, such as with respect to FIGS. 3A-3D, the interface surfaces can take a variety of form factors to control the orientation of the needle in the lumen. The surfaces of the needles that interface with inserts may be prepared in a variety of ways, such as, grinding, pressing, cold forming, stamping, machining, multidimensional printing, molding, casting, and the like. The manner in which the interface surfaces of the needles are formed may affect the needle lumen. For example, needle lumen 226-2 of needle 204-2 may be oblong due to the use of mechanical pressing to form the interface surfaces. However, needle lumen 226-1 of needle 204-1 may remain circular due to the use of grinding to form the interface surfaces. As shown in the illustrated embodiment, the lumens 214-1, 214-2 may have different diameters. In various embodiments, the diameters of one or more of the lumens may be selected to prevent kinking or maintain column strength.

FIGS. 3A-3D illustrate exemplary inserts 328A-1, 328A-2, 328B, 328C, 328D and needles 304B, 304C for a biopsy device according to one or more embodiments of the present disclosure. More specifically, FIG. 3A illustrates a transverse cross-sectional view of inserts 328A-1, 328A-2. Insert 328A-1 may include lumen interface 332A-1 and needle interface 334A-1 and insert 328A-2 may include lumen interface 332A-2 and needle interface 334A-2. FIG. 3B illustrates a transverse cross-sectional view of insert 328B in conjunction with needle 304B. Insert 328B may include lumen interface 332B and needle interface 334B and needle 304B may include insert interface 330B and needle lumen 326B. FIG. 3C illustrates a transverse cross-sectional view of insert 328C in conjunction with needle 304C. Insert 328C may include lumen interface 332C and needle interface 334C and needle 304B may include insert interface 330B and needle lumen 326B. FIG. 3D illustrates a transverse cross-sectional view of insert 328D. Insert 328D may include lumen interface 332D and needle interface 334D. In some embodiments, FIGS. 3A-3D may include one or more components that are the same or similar to one or more other components of the present disclosure. Further, one or more components of FIG. 3A-3D, or aspects thereof, may be incorporated into other embodiments of the present disclosure without departing from the scope of this disclosure. For example, the needle 304C along with insert 328C may be incorporated into biopsy device 100 without departing from the scope of this disclosure. Embodiments are not limited in this context.

In various embodiments, the inserts 328 in conjunction with features of a corresponding needle (e.g., insert 328B and needle 304B) may function to maintain an orientation (e.g., rotational orientation of the needle as it is moved proximally and distally in within a lumen of an elongate member). For example, a needle interface of each insert may mate with an insert interface of a corresponding needle. In some embodiments, a needle may be limited to a range of orientations by the insert. For example, a needle may be able to rotate five degrees within a lumen. Additionally, each insert may include a lumen interface for contacting a wall of a lumen when disposed in the lumen.

More generally, interface surfaces may take any shape, contour, or pattern. For example, an interface surface may include one or more curved, angular, and/or straight surfaces. In many embodiments, the interface surfaces may maintain a substantially uniform cross-sectional shape. In many such embodiments, the substantially uniform transverse cross-sectional shapes may allow corresponding interfaces to move proximally or distally with respect to one another. In several embodiments, changes in the transverse cross-sectional shape may be utilized to implement limits on proximal or distal motion of a needle. In some embodiments, corresponding interfaces may interlock.

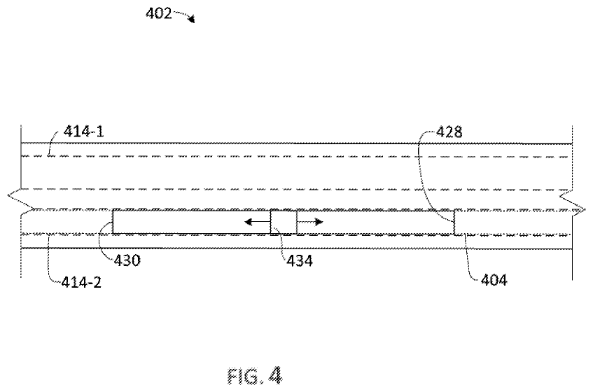

FIG. 4 illustrates various aspects of an elongate member 402 for a biopsy device according to one or more embodiments of the present disclosure. More specifically, FIG. 4 includes a side cross-section view of elongate member 402 with lumens 414-1, 414-2. The illustrated embodiment demonstrates limiting the proximal and/or distal motion of needle 404 with insert 428, such as due to a change in a transverse cross-sectional shape of the insert interface 430 and/or needle interface 434. In some embodiments, FIG. 4 may include one or more components that are the same or similar to one or more other components of the present disclosure. Further, one or more components of FIG. 4, or aspects thereof, may be incorporated into other embodiments of the present disclosure without departing from the scope of this disclosure. For example, insert 428 and needle 404 may be the same or similar to insert 228-1 and needle 204-1. Embodiments are not limited in this context.

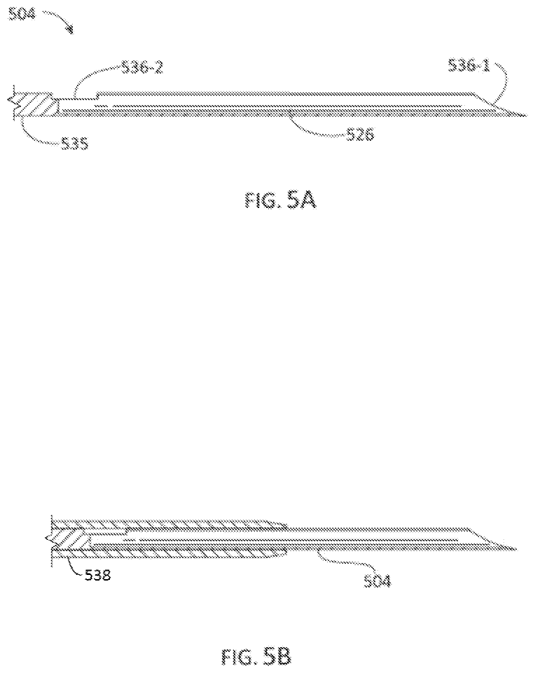

FIGS. 5A and 5B illustrate various aspects of a needle 504 for a biopsy device according to one or more embodiments of the present disclosure. More specifically, FIG. 5A includes a needle 504 having a solid bore 535 and a needle lumen 526 with openings 536-1, 536-2 distal of the solid bore 535. In the illustrated embodiment, the openings 536-1, 536-2 may be orthogonal with respect to each other. FIG. 5B includes needle 504 with a sheath 538 disposed around a portion of the needle. In some embodiments, FIGS. 5A and 5B may include one or more components that are the same or similar to one or more other components of the present disclosure. Further, one or more components of FIGS. 5A and 5B, or aspects thereof, may be incorporated into other embodiments of the present disclosure without departing from the scope of this disclosure. For example, needle 504 may be incorporated into biopsy device 100 without departing from the scope of this disclosure. Embodiments are not limited in this context.

The solid bore (or core) of the needle 504 may result in a robust needle that has increased column strength and increase flexural strength when compared to hollow bore needles. This can allow needle 504 to me inserted and removed from a lumen (e.g., of an ultrasound catheter or dual lumen catheter) without damaging the needle. In various embodiments, the opening 536-2 may allow air to escape from the needle when a sample is being acquired, preventing a pressure build up within the needle lumen 526 that prevents or limits the ability to acquire and retain a tissue sample therein. Further, the opening 536-2 may facilitate removal of a sample from the needle lumen 526. For example, a stylet may be inserted via opening 536-2 to expel a sample. In another example, a positive pressure with the proximal portion of the needle lumen 526 may be created via opening 536-2 to expel a sample.

The sheath 538 of FIG. 5B may comprise a vapor seal sheath that encloses a portion of the needle 504. In various embodiments, the sheath 538 may provide a vacuum in the needle lumen 526 as the needle 504 is withdrawn from a sample site. The vacuum created may retain larger samples in the needle lumen 526, thereby providing an improved and repeatable sampling process for eccentric nodules. In some embodiments, the sheath 538 may include a valve, such as a one-way valve. For example, sheath 538 may include a one-way valve to allow fluid to escape from opening 536-2, avoiding a pressure build up within the needle lumen 526 that prevents or limits the ability to acquire and retain a tissue sample therein.

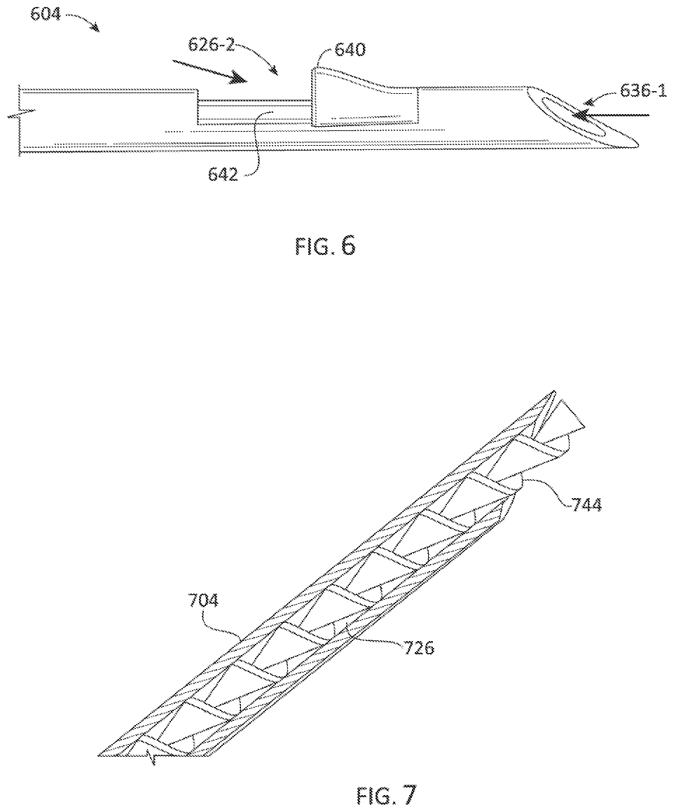

FIG. 6 illustrates a distal portion of needle 604 for a biopsy device according to one or more embodiments of the present disclosure. More specifically, FIG. 6 includes a side view of needle 604 with openings 636-1, 636-2, scoop 640, and tray 643. In many embodiments, scoop 640 may enable multidirectional sample acquisition. For example, opening 636-1 at the distal end of needle 604 may acquire a first tissue sample as the needle moves proximally into a target site, and the scoop 640 may sheer a second tissue sample into the tray 642 via opening 636-2 as the needle moves distally out of the target site. In some embodiments, multidirectional sample acquisition may include linear and/or rotational movements for sample acquisition. . In some embodiments, FIG. 6 may include one or more components that are the same or similar to one or more other components of the present disclosure. Further, one or more components of FIG. 6, or aspects thereof, may be incorporated into other embodiments of the present disclosure without departing from the scope of this disclosure. For example, insert 428 and needle 404 may be the same or similar to insert 228-1 and needle 204-1. Embodiments are not limited in this context.

In some embodiments, the scoop 640 may transition between a collapsed configuration and a deployed configuration. In several embodiments, FIG. 6 may illustrate a scoop in the deployed configuration. On the other hand, in the collapsed configuration one or more portions of the scoop 640 may move into the tray 642. For example, in the collapsed configuration scoop 640 may fit within the outer diameter of the cylindrical portion of the needle 604.

In many embodiments, scoop 640 may be biased into the expanded configuration. In many such embodiments, scoop 640 may transition into the collapsed configuration due to pressure on the proximal surface of the scoop 640. For example, the scoop 640 may transition into the collapsed configuration as it penetrates a target tissue in the distal direction. In such examples, the scoop 640 may return to the deployed configuration prior to, or in response to, removal from the target tissue in the proximal direction. Accordingly, in some embodiments, scoop 640 may comprise a shape-memory material.

FIG. 7 illustrates a distal portion of needle 704 for a biopsy device according to one or more embodiments of the present disclosure. More specifically, FIG. 7 includes a side cross-sectional view of needle 704 having a needle lumen 726 with a helical element 744 disposed therein. In several embodiments, helical component 744 may sheer off and draw sample tissue into needle lumen 726. For instance, helical component 744 may function similar to a water screw. In many embodiments, the helical component may be rotated by a power source. In one or more embodiments, the power source may also be utilized to rotate an imaging transducer, such as during radial ultrasound imaging with ultrasound catheter 106. In some embodiments, FIG. 7 may include one or more components that are the same or similar to one or more other components of the present disclosure. Further, one or more components of FIG. 7, or aspects thereof, may be incorporated into other embodiments of the present disclosure without departing from the scope of this disclosure. For example, the solid bore of needle 504 may be incorporated into needle 704 without departing from the scope of this disclosure. Embodiments are not limited in this context.

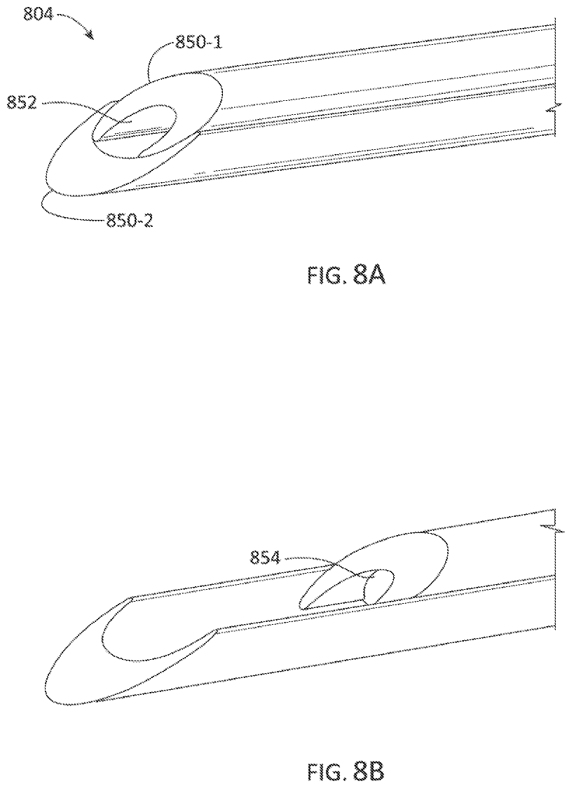

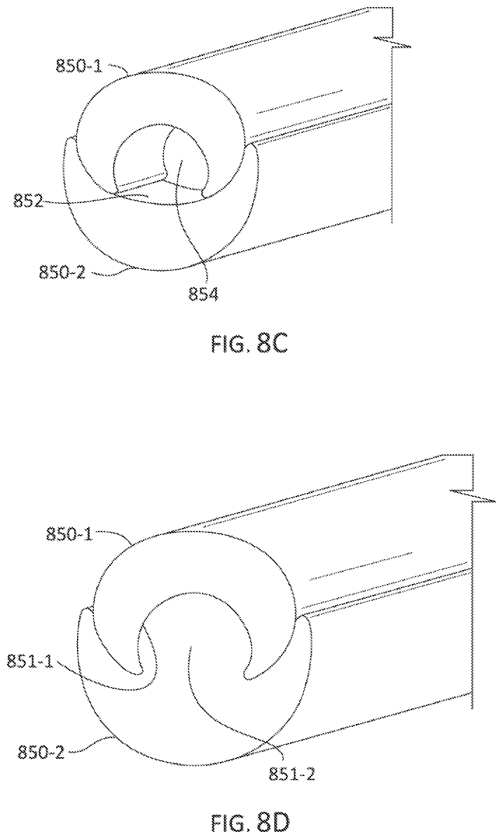

FIGS. 8A-8D illustrate various aspects of a needle 804 for a biopsy device according to one or more embodiments of the present disclosure. More specifically, FIG. 8A illustrates a distal portion of needle 804 with components 850-1, 850-2 fit together in a penetration configuration with needle cavity 852. FIG. 8B illustrates a base 854 of the needle cavity 852 and the components 850-1, 850-2 fit together in a sample removal configuration. Accordingly, one or more of components 850-1, 850-2 may move linearly with respect to each other (similar to inserts and needles). FIG. 8C illustrates a transverse cross-sectional view of the needle cavity 852 proximate cavity base 584. In some embodiments, needle 804 may transfer to a solid bore proximal of the cavity base 854. FIG. 8D illustrates a transverse cross-sectional view of needle 804 with component 850-1 including interlocking feature 851-1 and component 850-2 including interlocking feature 851-2. In many embodiments, the interlocking feature 851-2 may comprise the solid bore. The interlocking features 851-1, 851-2 may enable the components 850 to move linearly with respect to each other while preventing separation of the components absent moving the proximal end of one component past the distal end of the other component. In some embodiments, FIGS. 8A-8D may include one or more components that are the same or similar to one or more other components of the present disclosure. Further, one or more components of FIG. 8A-8D, or aspects thereof, may be incorporated into other embodiments of the present disclosure without departing from the scope of this disclosure. For example, aspects of the insert interface 430 of needle 404 mating with needle interface 434 of needle 404 may be incorporated into needle 804 to limit proximal and/or distal movement of components 850-1, 850-2 with respect to each other. In another example, the transverse cross-sectional shape of interlocking features 851-1, 851-2 may be incorporated into a corresponding insert needle pair without departing from the scope of this disclosure. Embodiments are not limited in this context.

The medical devices of the present disclosure are not limited to bronchoscopes, and may include a variety of medical devices for accessing body passageways, including, for example, catheters, ureteroscopes, duodenoscopes, colonoscopes, arthroscopes, cystoscopes, hysteroscopes, and the like. Further, in some embodiments, reference to endoscopy, endoscopic, endoscope etc. may generally refer to any medical device inserted into a body lumen. In one or more embodiments, a body passageway may be accessed for a biopsy procedure. For instance, a bronchoscope may be inserted into a patient for a lung nodule biopsy procedure (the location of the lung nodule may have been previously determined, such as based on virtual mapping and/or radiology). Once the bronchoscope is positioned, the medical biopsy device may be inserted through a working channel and out past the distal end of the bronchoscope (e.g., 15 centimeters).

In some embodiments, an imaging transducer may then be activated inside the airway to provide real-time imaging of the lung nodule. Based on real-time imaging of the lung nodule and the marker indications, the medical imaging device may be positioned to biopsy the lung nodule. Once positioned, the biopsy needle may be actuated one or more times to take one or more core samples within the hollow biopsy needle. Further, suction and aspiration through the needle may be used to remove the sample(s) from the hollow biopsy needle. Additionally, one or more steps of this process may be repeated as necessary in the same or other locations of the nodule, and/or in other locations of the same lung airway or of other airways of the lungs.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A biopsy device, comprising:
an elongate member having a proximal portion, a distal portion, and a lumen, wherein the lumen includes proximal and distal openings and extends from the proximal portion to the distal portion of the elongate member; and
an insert disposed in the lumen, the insert comprising a lumen interface and a needle interface, wherein the insert is configured to mate with an insert interface of a needle disposed in the lumen and maintain a rotational orientation of the needle.

2. The biopsy device of claim 1, wherein the insert limits proximal or distal motion of the needle disposed in the lumen.

3. The biopsy device of claim 1, wherein the needle interface comprises one or more of a flat surface, a channel, a groove, and a zig-zag.

4. The biopsy device of claim 1, comprising the needle, the needle including a needle lumen with first and second openings, wherein the first and second openings are in a distal half of the needle.

5. The biopsy device of claim 4, wherein the first opening of the needle lumen is orthogonal to the second opening of the needle lumen.

6. The biopsy device of claim 5, comprising a sheath disposed around a portion of the needle, wherein the sheath covers the first opening of the needle lumen.

7. The biopsy device of claim 4, wherein the needle comprises a scoop disposed adjacent to the first opening.

8. A system, comprising:
an elongate member having a proximal portion, a distal portion, and a lumen, wherein the lumen includes proximal and distal openings and extends from the proximal portion to the distal portion of the elongate member;
an insert disposed in the lumen, the insert comprising a lumen interface and a needle interface; and
a needle disposed in the lumen, the needle comprising an insert interface,
wherein the needle interface and the insert interface include corresponding mating features.

9. The system of claim 8, comprising a stylet disposed in a lumen of the needle.

10. The system of claim 9, wherein the stylet comprises a polymer.

11. The system of claim 8, the needle comprising a needle lumen with first and second openings, wherein the first and second openings are in a distal half of the needle.

12. The system of claim 11, wherein the first opening of the needle lumen is orthogonal to the second opening of the needle lumen.

13. A method, comprising:
forming an insert with a needle interface;
forming a needle with an insert interface, wherein the needle interface of the insert corresponds to the insert interface of the needle;
mating the needle interface with the insert interface; and
disposing the insert and the needle in a lumen of an elongate member.

14. The method of claim 13, comprising:
forming a stylet from a polymer; and
disposing the stylet in a lumen of the needle.

15. The method of claim 13, comprising press-fitting the insert into the lumen.

16. The method of claim 13, comprising grinding the needle to form the insert interface.

17. The method of claim 13, comprising pressing the needle to form the insert interface.

18. The method of claim 13, comprising forming the insert by stamping.

19. The method of claim 13, comprising disposing the insert and the needle in the lumen with press fitting.

20. The method of claim 13, comprising disposing the insert and the needle in the lumen with thermal fitting.
